(19) United States
(12) Patent Application Publication
(10) Pub. No.: US 2002/0044953 A1
(43) Pub. Date: Apr. 18, 2002

(54) Use of non-prostanoic agonists of prostaglandin EP-2 and/or EP-4 receptors as cosmetic agents for attenuating, reducing or stopping the loss of head hair and other hairs

(76) Inventors: Jean Francois Michelet, Creteil (FR); Yann Mahe, Morsang-Sur-Orge (FR); Bruno Bernard, Neuilly-Sur-Seine (FR)

Correspondence Address: JACOBSON HOLMAN, PROFESSIONAL LIMITED LIABILITY COMPANY, 400 SEVENTH STREET, N.W., WASHINGTON, DC, 20004, US

(21) Appl. No.: 09/917,236

(22) Filed: Jul. 30, 2001

(30) Foreign Application Priority Data
Jul. 28, 2000 (FR) 00 09981

(51) Int. Cl. A61K 31/557; A61K 7/00

(52) U.S. Cl. 424/401; 514/573

(57) ABSTRACT

The invention relates to the use of non-prostanoic agonists of prostaglandin EP-2 and/or EP-4 receptors as cosmetic agents for attenuating, reducing or stopping the loss of head hair and other hairs.

The invention relates to the use of non-prostanoic agonists of prostaglandin EP-2 and/or EP-4 receptors as cosmetic agents for attenuating, reducing or stopping loss of head hair and other hairs.

Man has a complement of 100 000 to 150 000 hairs and it is normal to use 50 to 100 hairs daily. Maintenance of this complement results essentially from the fact that the life of a hair is subject to a hair cycle in the course of which the hair forms, grows and falls out, before being replaced with a new hair which appears in the same follicle.

Three phases are observed in the course of a hair cycle, namely: the anagenic phase, the catagenic phase and the telogenic phase.

In the course of the first phase, known as the anagenic phase, the hair passes through a period of active growth associated with intense mitotic activity at the bulb.

The second phase, known as the catagenic phase, is transient and is marked by an interruption of the mitotic activity of the bulb. During this phase, the hair undergoes an involution, the follicle becomes atrophied and its dermal implantation moves upwards.

The end phase, known as the telogenic phase, corresponds to a resting period of the follicle and the hair finishes by falling out. After this resting phase, a new follicle is regenerated, in the place of the previous one.

This process of permanent physical renewal undergoes a natural evolution in the course of ageing, the hairs become finer and their cycles shorter (M. Courtois et al., 1995, Br. J. Dermatol., 132: 86-93).

In almost all cases, hair loss occurs in genetically predisposed individuals; it more particularly affects men.

This hair loss occurs when the process of physical renewal is accelerated or disrupted, i.e. the growth phases are shortened (Mr Courtois et al., 1994, Skin Pharmacol., 7: 84-89), the hairs pass to the telogenic phase earlier and they fall out in larger numbers. The successive growth cycles result in increasingly fine and increasingly short hairs, which become converted gradually into an unpigmented down. This phenomenon may lead to baldness.

Compositions for preventing or reducing hair loss and optionally for inducing or stimulating hair growth have been sought for many years in the cosmetic or pharmaceutical industry.

In this perspective, compounds such as 6-1-(piperidyl)-2,4-pyrimidinediamine 3-oxide or “Minoxidil” have been used. The use of a lotion containing an azole derivative and most specifically 1-acetyl-4-{4-[2-(2,4-di-chlorophenyl)-2-(1H-imidazol-1-ylmethyl)-1,3-dioxolan-4-ylmethoxy]phenyl}piperazine for the treatment of alopecia is described in patent WO 92/00057.

In parallel, the article “Growth regulation of primary human keratinocytes by prostaglandin E receptor EP₂ and EP₃ subtypes” by Konger et al. (Biochimica Biophysica Acta, 1401, 1998, 221-224) describes that prostaglandin receptors play an important role in regulating the growth of epidermal keratinocytes. It is also shown in the said article that prostanoic agonists of these prostaglandin receptors, for instance 11-deoxy PGE₁ induce a stimulation of epidermal keratinocyte growth.

Nevertheless, it is well known that the programmes of differentiation of the keratinocytes of the epidermis and of hair follicles are clearly different. Thus, it is known that differentiation markers such as keratins K1 and K10 are not expressed in hair follicles and in particular in the outer sheath (Lenoir et al., 1988, Dev. Biol. 130: 610-620); that trichohyalin is expressed in hair follicles, in particular in the inner sheath but not in the epidermis (O'Guin et al., 1992, J. Invest. Dermatol. 98: 24-32); and that type 1 cyclooxygenase is not expressed in the keratinocytes of hair follicles but is expressed in the epidermis (Michelet et al., 1997, J. Invest. Dermatol. 108: 205-209).

Furthermore, it is known that the keratinocytes of the epidermis and of hair follicles behave differently in response to the same pharmacological agent. Thus, it is known that, in vivo, treating the epidermis with retinoic acid induces hyperplasia and spongiosis (Griffiths et al., 1993, J. Invest. Dermatol. 101: 325-328) whereas treating the scalp induces a loss of hair (Berth-Jones et al., 1990, Br. J. Dermatol. 122: 75-755), and that, in vitro, retinoic acid, depending on the dose used, promotes or reduces the differentiation of the epidermis (Asselineau et al., 1989, Dev. Biol. 133: 32-335), while it causes an interruption of growth of the hair follicles (Billoni et al., 1997, Acta Dermatol. Venerol. 77: 350-355). It is also known that EGF induces epidermal hyperplasia and, simultaneously, regression of the hair follicles (Philip et al., 1985, J. Invest. Dermatol. 84: 172-175).

Patent WO 98/33497 describes pharmaceutical compositions containing prostaglandins or prostaglandin derivatives which act as prostanoic agonists of the prostaglandin receptors in order to combat hair loss in man. In the said document, prostanoic agonists of the type A₂, F₂α and E₂ are preferred for treating hair loss.

The Applicant has now discovered that by using non-prostanoic agonists of the prostaglandin EP-2 and/or EP-4 receptors, a large induction and large stimulation in the growth of head hair and other hairs and strong action on slowing down the loss of head hair and other hairs are found, surprisingly.

The Applicant has thus found that the use in accordance with the invention makes it possible to obtain a rapid effect, at a low concentration and/or with a low rate of application.

Furthermore, the non-prostanoic agonists of the prostaglandin EP-2 and/or EP-4 receptors of the invention are particularly of low toxicity and show good conservation.

The use of these agonists makes it possible to obtain, in particular compared with those of the prior art, more effective compositions which may be used in particularly easy manner, and which also allow the compositions to be removed easily by simple rinsing.

The compounds in accordance with the invention are moreover particularly suitable in cosmetic terms and do not cause any irritation of the scalp, even after prolonged contact, without rinsing.

Thus, one subject of the invention is the use of non-prostanoic agonists of prostaglandin EP-2 and/or EP-4 receptors as cosmetic agents for attenuating, reducing or stopping the loss of head hair and other hairs.

These compounds make it possible to prevent or reduce the loss of head hair and other hairs and optionally to induce or stimulate the growth of head hair and other hairs.

A subject of the invention is also the use of a non-prostanoic agonist of prostaglandin EP-2 and/or EP-4 receptors in a cosmetic composition and also in a cosmetic treatment process for attenuating, reducing or stopping the loss of head hair and other hairs.

The main subject of the invention is a cosmetic or dermatological composition containing at least one non-prostanoic agonist of prostaglandin EP-2 and/or EP-4 receptors in a cosmetically or dermatologically acceptable medium.

The prostaglandin EP-2 and/or EP-4 receptors are receptors of prostaglandins of the E2 series. These receptors combine a family of 4 major representatives (EP1, EP2, EP3 and EP4) and have very varied tissue activities.

The prostaglandins are biological effectors derived from polyunsaturated fatty acid such as, for example, arachidonic acid for PGA₂, PGE₂, PGF₂α and TXA₂, or from dihomo-γ-linolenic acid for PGE₁. The prostaglandins are involved in many physiological regulation phenomena. Prostanoic agonists of prostaglandin receptors are described in the article “Prostanoid Receptors: Structure, Properties and Functions” by Shush Narumyia et al., Physiological review, Vol. 79, 1999, 1193-1226. These prostanoic agonists have in common a cyclopentane moiety of the type I:

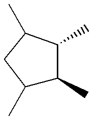

An agonist is a compound which binds to a receptor and which induces a biological response similar to that obtained with the natural ligand which activates this response.

The expression “non-prostanoic agonist of prostaglandin EP-2 and/or EP-4 receptors” means a compound not comprising a cyclopentane ring of the type I, for attenuating, reducing or stopping the loss of head hair and other hairs. These agonists are capable of preventing or reducing the loss of head hair and other hairs and possibly of stimulating the growth of head hair and other hairs.

The term “other hairs” also means the eyelashes, the eyebrows and any hairs in general.

According to the invention, the said cosmetic composition may contain from 0.001% to 10% and preferably from 0.01% to 5% of non-prostanoic agonists of prostaglandin EP-2 and/or EP-4 receptors by weight relative to the weight of the composition.

It is also possible to use in addition other cosmetic agents for stopping hair loss and/or increasing the growth of head hair and other hairs in the cosmetic compositions defined above, such as, for example, prostaglandin EP-3 receptor antagonists in proportions ranging from 0.001% to 10% and preferably from 0.1% to 5% of antagonists by weight relative to the weight of the composition, or alternatively compounds known for their properties on the loss and/or growth of head hair and/or other hairs, such as, for example, Minoxidil or 2,4-diaminopyrimidine 3-oxide or Aminexil.

The physiologically acceptable medium used for the compositions of the invention is a medium which can consist of water or a mixture of water and a solvent or a mixture of solvents. The solvents are chosen from acceptable organic solvents chosen more particularly from C1-C4 lower monofunctional or polyfunctional alcohols, for instance ethanol, isopropanol, tert-butanol, optionally oxyethylenated polyethylene glycols, polypropylene glycol esters, sorbitol and its derivatives, dialkyl isosorbides, glycol ethers and propylene glycol ethers, and fatty esters.

When they are present, the solvents are present in proportions of between 5% and 98% by weight relative to the total weight of the composition.

The composition may in addition contain a fatty phase. In this case, the fatty phase represents 0% to 50% of the total weight of the composition.

These compositions may also contain:

esterified oligosaccharides such as those described in EP-A-0 064 012;

hexosaccharic acid derivatives such as those described in EP-A-0 375 388, in particular glucosaccharic acid;

glycosidase inhibitors such as those described in EP-A-0 334 586, in particular D-glycaro-1,5-lactam;

glycosaminoglycanase and proteoglycanase inhibitors such as those mentioned in EP-A-0 277 428, in particular L-galactano-1,4-lactone;

tyrosine kinase inhibitors such as those described in EP-A-0 403 238, in particular 1-amido-1-cyano-(3,4-dihydroxyphenyl)ethylene;

hyperaemiants such as:

nicotinic acid esters including, more particularly, benzyl and C₁-C₆ alkyl nicotinates and in particular methyl and benzyl nicotinate, and also tocopheryl nicotinate;

xanthine bases including, more particularly, caffeine and theophylline;

capsaicin;

UV-A and UV-B screening agents, for instance methoxycinnamates and benzophenone derivatives;

phosphodiesterase inhibitors such as Visnadine®;

adenine cyclase activators such as Forskolin;

antioxidants and free-radical scavengers, in particular

for OH radicals such as DMSO;

α-tocopherol, BHA and BHT;

superoxide dismutase (SODIUM);

antidandruff agents such as omadine and octopirox;

moisturizers such as urea, glycerol, lactic acid,

α-hydroxy acids, thiamorpholinone and its derivatives, and lactones;

antiseborrhoeic agents such as S-carboxymethylcysteine, S-benzylcysteamine and derivatives thereof, and thioxolone;

antiandrogens and hormones such as oesrtiol, oestradiol, thyroxine, oxendolone and diethylstilbestrol;

retinoids including, more particularly, t-trans-retinoic acid, also known as tretinoin, isotretinoin, retinal or vitamin A and its derivatives, such as the acetate, palmitate or propionate, motretinide, etretinate and zinc t-trans-retinoate;

antibacterial agents chosen, more particularly, from Irgasan, macrolides, pyranosides and tetracyclines, and in particular erythromycin;

calcium antagonists, among which mention may be made of Cinnarizine and Diltiazem as non-limiting examples;

phospholipids such as lecithin;

diazoxide (3-methyl-7-chloro-1,2,4-[2H]benzothiadiazine 1,1-dioxide);

linoleic and linolenic acids;

anthralin and its derivatives;

5-alkanol salicylic acid and its derivatives as described in patent FR-2 581 542;

penetration activators such as THF, 1,4-dioxane, oleic acid, 2-pyrrolidone, benzyl salicylate, etc.,

vitamins or provitamins such as β-carotene, biotin, panthenol and its derivatives, vitamin C and vitamins B₂, B₄ and B₆.

These compositions may also contain cyclic AMP.

These compositions may also additionally contain preserving agents, stabilizers, pH regulators, osmotic pressure modifiers, emulsifiers and conventional hydrophilic or lipophilic gelling agents and/or thickeners; hydrophilic or lipophilic active agents; preserving agents; antioxidants; fragrances; emulsifiers; moisturizers; pigmenting agents; depigmenting agents; keratolytic agents; vitamins; emollients; sequestering agents; surfactants; polymers; acidifying or basifying agents; fillers; free-radical scavengers; ceramides; sunscreens; insect repellents; slimming agents; dyestuffs; bactericides; antidandruff agents.

The compositions in accordance with the invention may also contain surfactants including, in particular, those chosen from nonionic and amphoteric surfactants.

Among the nonionic surfactants, those which will be mentioned are the polyhydroxypropyl ethers described in particular in French patents Nos. 1 477 048; 2 091 516; 2 169 787; 2 328 763; 2 574 786; oxyethylenated (C₈-C₉)alkylphenols comprising from 1 to 100 mol of ethylene oxide and preferably 5 to 35 mol of ethylene oxide; alkylpolyglycosides of formula: CₙH₂ₙ₊₁ (C₆H₁₀O₅)ₓH in which n ranges from 8 to 15 inclusive and x from 1 to 10 inclusive.

Among the amphoteric surfactants, those which will be mentioned more particularly are the amphocarboxyglycinates and amphocarboxypropionates defined in the CTFA dictionary, 3rd edition, 1982, and sold in particular under the name Miranol® by the company Miranol.

Cationic and/or anionic surfactants may also be used.

The compounds in accordance with the invention may also be introduced into gelled or thickened supports, such as essentially aqueous supports gelled with heterobiopolysaccharides, such as xanthan gum, scleroglucans or cellulose derivatives, in particular cellulose ethers, aqueous-alcoholic supports gelled with polyhydroxyethyl acrylates or methacrylates or essentially aqueous supports thickened in particular with polyacrylic acids crosslinked with a polyfunctional agent, such as the Carbopols sold by the company Goodrich.

The thickeners are preferably present in proportions of between 0.05% and 5% by weight and in particular between 0.2% and 3% by weight relative to the total weight of the composition.

Needless to say, a person skilled in the art will take care to select the optional compound(s) to be added to the composition according to the invention, such that the advantageous properties intrinsically associated with the composition in accordance with the invention are not, or are not substantially, adversely affected by the addition envisaged.

The composition defined above may be in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase with the aid of spherules.

The composition may have a pH of between 3 and 8.

The composition may have the appearance of a white or colored cream, an ointment, a milk, a lotion, a serum, a paste, a mousse or a solid.

These compositions defined above may be applied to the hair or the scalp and can be applied, for example, after washing the scalp and the hair with a shampoo.

A subject of the invention is also the use of non-prostanoic agonists of the prostoglandin EP-2 and/or EP-4 receptors as cosmetic or dermatological agents for attenuating, reducing or stopping the loss of head hair and other hairs.

A subject of the invention is also the use of a composition as defined above to attenuate, reduce or stop loss of head hair and other hairs. The agonists are used in accordance with the invention to prevent or reduce the loss of head hair and other hairs and possibly to stimulate the growth of head hair and other hairs.

Another subject of the invention is a cosmetic or dermatological treatment process for attenuating, reducing or stopping the loss of head hair and other hairs, which consists in applying to head hair or other hairs a cosmetically or dermatologically effective amount of non-prostanoic agonists of prostaglandin EP-2 and/or EP-4 receptors.

Another cosmetic treatment process for attenuating, reducing or stopping the loss of head hair and other hairs consists in applying to head hair or other hairs a cosmetic or dermatological composition as defined above.

The examples which follow are intended to illustrate the invention without, however, being limiting in nature.

### EXAMPLE 1

### Lotion for Preventing Hair Loss

Non-prostanoic agonist of prostaglandin

| | |
|---|---|
| EP-2 receptors | 0.5 g |
| Propylene glycol | 20 g |
| 95° Ethanol | 30 g |
| Water qs | 100 g |

This lotion is applied daily at a rate of 10 ml to the scalp for 2 to 3 months. A marked slowing down in the daily loss of head hair and other hairs is then observed.

### EXAMPLE II

### Shampoo For Preventing Hair Loss

Non-prostanoic agonist of prostaglandin

| | | |
|---|---|---|
| EP-4 receptors | 1.5 | g |
| Polyglyceryl 3-hydroxylauryl ether | 26 | g A.M. |
| Hydroxypropylcellulose sold under the | | |
| name Klucell G by the company Hercules | 2 | g |
| Prostaglandin EP-3 receptor antagonist | 1 | g |
| Preserving agent | qs | |
| 95° Ethanol | 50 | g |
| Aminexil | 0.1 | g |
| Water qs | 100 | g |

This shampoo is used daily at a rate of 15 g per head of hair, with an exposure time of about one minute, over a period of 4 months. An appreciable slowing down in the daily loss of hair is then observed.

### EXAMPLE III

### Gel For Preventing Hair Loss

Non-prostanoic agonist of prostaglandin

| | | |
|---|---|---|
| EP-2 receptors | 0.75 | g |
| Essential oil of eucalyptus | 1 | g |
| Econazole | 0.2 | g |
| Lauryl polyglyceryl 6 cetearyl glycol | 1.9 | g |
| ether | | |
| Sodium glutamate off hydrogenated tallow, | 0.1 | g |
| sold under the name Acylglutamate HS110 | | |
| by the company Ajinomoto | | |
| Preserving agents | | |
| Carbopol 934P sold by the company BF | 0.3 | g A.M. |
| Goodrich Corporation | | |
| Neutralizer | qs | pH 7 |
| Water qs | 100 | g |

This gel is applied twice a day (morning and evening) at a rate of 25 g to the entire scalp with final massaging. After application for 3 months, the daily loss of head hair and other hair is clearly slowed down.

### EXAMPLE IV

### Lotion For Preventing Hair Loss

Non-prostanoic agonist of prostaglandin

| | |
|---|---|
| EP-4 receptors | 0.4 g |
| Propylene glycol | 20 g |
| 95° Ethanol | 50 g |
| Aminexil | 0.1 g |
| Water qs | 100 g |

This lotion is used in the same way as in Example 1. The results observed are of the same order.

### Experiment

In order to study the behaviour of hair follicles in the presence of non-prostanoic agonists of prostaglandin EP-2 and/or EP-4 receptors, the Applicant used the “surviving hair” method from L'Oreal patent FR 9508465.

From a scalp biopsy, a fairly thin strip of scalp was isolated using a scalpel. With microtweezers, the adipose tissue around the follicles was removed, while taking care not to damage the hair bulb. Under a microscope, the follicle was cut away using a scalpel to separate it from its epidermal and dermal environment.

One of the fragments obtained was cultured in Williams E medium at 37° C. under a humid atmosphere in the presence of 5% CO₂ and was used as control.

The other fragments were placed in the same culture medium in the presence of non-prostanoic agonists of prostaglandin EP-2 and/or EP-4 receptors.

The fragments in the presence of the agonists thus maintained in cell culture extend in a significantly greater manner in comparison with the agonist-free control fragment.

1. Cosmetic composition containing at least one non-prostanoic agonist of prostaglandin EP-2 and/or EP-4 receptors in a cosmetically acceptable medium.

2. Composition according to claim 1, characterized in that the said cosmetic composition contains from 0.001% to 10% and preferably from 0.01% to 5% of agonists by weight relative to the weight of the composition.

3. Composition according to claim 1 or 2, characterized in that the said cosmetic composition contains from 0.001% to 10% and preferably from 0.1% to 5% of prostaglandin EP-3 receptor antagonists by weight relative to the weight of the composition.

4. Composition according to any one of claims 1 to 3, characterized in that the composition also contains a cosmetically acceptable medium consisting of water or of water and at least one organic solvent chosen from the group consisting of hydrophilic organic solvents, lipophilic organic solvents and amphiphilic organic solvents, or mixtures thereof.

5. Composition according to claim 4, characterized in that the organic solvents are chosen from the group consisting of monofunctional or polyfunctional alcohols, optionally oxyethylenated polyethylene glycols, polypropylene glycol esters, sorbitol and its derivatives, dialkyl isosorbides, glycol ethers and polypropylene glycol ethers, and fatty esters.

6. Composition according to claim 4 or 5, characterized in that the organic solvent(s) represent(s) from 5% to 98% of the total weight of the composition.

7. Composition according to any one of claims 1 to 6, characterized in that the composition comprises at least one fatty phase.

8. Composition according to claim 7, characterized in that the fatty phase represents from 0% to 50% of the total weight of the composition.

9. Composition according to any one of claims 1 to 8, characterized in that it contains at least one additive chosen from the group consisting of conventional hydrophilic or lipophilic gelling agents and/or thickeners; hydrophilic or lipophilic active agents; preserving agents; antioxidants; fragrances; emulsifiers; moisturizers; pigmenting agents; depigmenting agents; keratolytic agents; vitamins, emollients; sequestering agents; surfactants; polymers; acidifying or basifying agents; fillers; free-radical scavengers; ceramides; sunscreens; insect repellents; slimming agents; dyestuffs; bactericides; antidandruff agents.

10. Composition according to any one of claims 1 to 9, characterized in that the composition is in the form of an aqueous, aqueous-alcoholic or oily solution, an oil-in-water or water-in-oil or multiple emulsion, and aqueous or oily gel, a liquid, pasty or solid anhydrous product or a dispersion of oil in an aqueous phase using spherules.

11. Composition according to any one of claims 1 to 10, characterized in that the composition has the appearance of a white or colored cream, an ointment, a milk, a lotion, a serum, a paste, a mousse or a solid.

12. Composition according to any one of claims 1 to 11, characterized in that the composition has a pH of between 3 and 8.

13. Use of non-prostanoic agonists of prostaglandin EP-2 and/or EP-4 receptors, as cosmetic agents for attenuating, reducing or stopping the loss of head hair and other hairs.

14. Use of a composition described in any one of claims 1 to 12 for attenuating, reducing or stopping the loss of head hair and other hairs.

15. Cosmetic treatment process for attenuating, reducing or stopping the loss of head hair and other hairs, characterized in that it consists in applying to head hair or other hairs a cosmetically effective amount of non-prostanoic agonists of prostaglandin EP-2 and/or EP-4 receptors.

16. Cosmetic treatment process for attenuating, reducing or stopping the loss of head hair and other hairs, characterized in that it consists in applying to the head hair or other hairs a cosmetic composition as defined in any one of claims 1 to 12.
